(19) United States
(12) Patent Application Publication
(10) Pub. No.: US 2024/0374268 A1
(43) Pub. Date: Nov. 14, 2024

(54) DEVICES AND METHODS FOR REMOVING MATERIAL FROM A PATIENT

(71) Applicant: Penumbra, Inc., Alameda, CA (US)

(72) Inventor: Michael Schaller, Louisville, CO (US)

(73) Assignee: Penumbra, Inc., Alameda, CA (US)

(21) Appl. No.: 18/601,628

(22) Filed: Mar. 11, 2024

Related U.S. Application Data
(60) Provisional application No. 63/466,284, filed on May 13, 2023.
(60) Provisional application No. 63/524,788, filed on Jul. 3, 2023.

(51) Int. Cl. A61B 17/22; A61B 17/00; A61B 90/00

(52) U.S. Cl. CPC A61B 17/22; A61B 2017/00154; A61B 2017/00292; A61B 2017/00398; A61B 2017/0042; A61B 2017/22079; A61B 2090/066; A61B 2217/005

(57) ABSTRACT

Devices for removing occlusive material from blood lumens and similar physiological structures are provided and generally include a progressive cavity pump. The progressive cavity pump may be integrated into a handle assembly or a distal end of a catheter and may be drive by a motor activated by a controller. In certain implementations, the controller may be configured to activate the motor to generate a pulsatile vacuum using the progressive cavity pump. To facilitate activation of the motor by the controller, the device may further include a pressure sensor configured to measure pressure distal the progressive cavity pump.

### CROSS-REFERENCE TO RELATED APPLICATIONS

This application claims the priority benefit of U.S. Provisional Patent Application No. 63/466,284, filed May 13, 2023, and titled “Treatment Device and Method” and of U.S. Provisional Patent Application No. 63/524,788, filed Jul. 3, 2023, and titled “Treatment Device and Method” which are all herein incorporated by reference in their entirety.

### GOVERNMENT LICENSE RIGHTS

This invention was made with government support under R41 NS129430 awarded by the National Institutes of Health. The government has certain rights in the invention.

### TECHNICAL FIELD

This disclosure relates generally to the field of medicine, and more specifically to the field of interventional radiology. Described herein are devices and methods for removing unwanted materials from a patient.

### BACKGROUND

The removal of materials from patients is an important part of routine and emergency medical care. For example, in the field of interventional radiology, removal of clot (which includes thrombus or thromboemboli) from blood vessels or artificial vascular grafts is known as thrombectomy and a variety of devices have been proposed to address this need. Limitations of some existing devices include but are not limited to significant amounts of blood loss during clot extraction, difficulty removing a variety of clot compositions including both soft and hard clot, difficulty removing clot adhered to the vessel wall, injury to blood vessels during clot extraction, large device size, clot fragmentation with subsequent embolization during removal, and the amount of capital equipment required to operate the devices. A device that can remove materials (e.g., clots, stones, malignant tissues) from body lumens or cavities which overcomes some or all of these limitations would be advantageous.

### SUMMARY

In one aspect of this disclosure, a device is provided and includes a handle body and a progressive cavity pump disposed within the handle body. The progressive cavity pump includes a stator and a rotor and is configured to transfer fluid between a first volume within the handle body and a second volume within the handle body. The device further includes a motor coupled to the rotor and configured to drive the rotor, a pressure sensor in communication with the first volume and configured to measure pressure within the first volume, and a controller communicatively coupled to the motor and configured to activate the motor based on pressure measurements obtained from the pressure sensor.

In another aspect of this disclosure, a device is provided that includes a handle body, a progressive cavity pump disposed within the handle body, a motor coupled to drive a rotor of the progressive cavity pump; and a controller communicatively coupled to the motor and configured to activate the motor in response to each of activation of a throttle assembly actuatable by a user of the device and pressure measurements obtained from a pressure sensor in communication with a volume distal the progressive cavity pump.

In yet another aspect of the present disclosure, a device is provided that includes a handle body and a progressive cavity pump disposed within the handle body. The progressive cavity pump includes a stator and a rotor and is configured to transfer fluid between a first volume within the handle body and a second volume within the handle body. The device further includes a motor coupled to the rotor and configured to drive the rotor, a pressure sensor in communication with the first volume and configured to measure pressure within the first volume, and a controller communicatively coupled to the motor. The controller is configured to activate the motor in response to each of activation of a throttle assembly actuatable by a user of the device and pressure measurements obtained from the pressure sensor.

In another aspect of the present disclosure, a device is provided that includes a handle coupleable to a catheter, a motor, and a progressive cavity pump. The progressive cavity pump includes a rotor operably coupled to the motor such that actuation of the motor causes rotation of the rotor. When the handle is coupled to the catheter, the progressive cavity pump is in fluid communication with the catheter. The device further includes a controller communicatively coupled to the motor and configured to activate the motor to produce a pulsatile vacuum using the progressive cavity pump.

In another aspect of the present disclosure, a method of removing occlusive material within a blood lumen of a patient is provided. The method includes disposing a catheter coupled to a handle assembly within the blood lumen and generating a pulsatile vacuum using a progressive cavity pump in fluid communication with the catheter and in a volume distal the progressive cavity pump.

### BRIEF DESCRIPTION OF THE DRAWINGS

FIG. 1A illustrates an implementation of a device according to the present disclosure in an isometric view.

FIG. 1B illustrates an exploded component view of the device of FIG. 1A.

FIG. 2 illustrates a rotor and torque member of the device of FIG. 1A in a section view.

FIG. 3A illustrates an implementation of the rotor with a lumen.

FIG. 3B illustrates another implementation of the rotor with an alternative lumen.

FIG. 3C illustrates another implementation of the rotor with another alternative lumen.

FIG. 4 illustrates another implementation of the device including a pumping assembly within the handle assembly.

FIG. 5 illustrates the handle assembly of FIG. 4 with an aspiration catheter and hemostasis valve.

FIG. 6 illustrates a section view of the handle assembly of FIG. 4.

FIG. 7 illustrates another implementation of the pumping assembly within the handle assembly.

FIG. 8 illustrates an implementation of a vacuum profile of the device.

FIG. 9 illustrates another implementation of a vacuum profile of the device.

FIG. 10 illustrates another implementation of a vacuum profile of the device.

### DETAILED DESCRIPTION

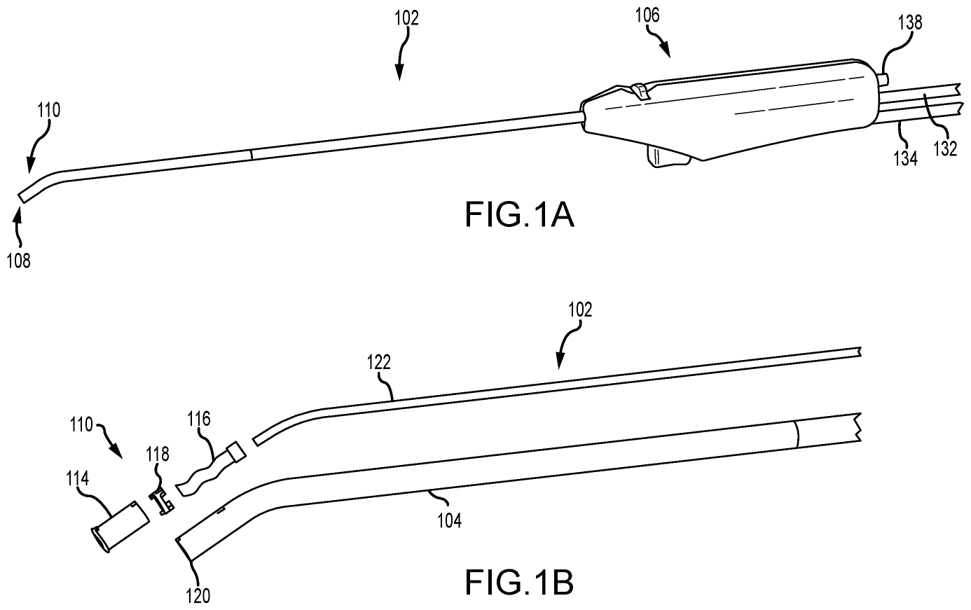

In FIG. 1A, a material removal device 102 is shown in isometric view. The device 102 may be similar to and/or include aspects of the removal devices that have been described in PCT application PCT/US2023/063580 (the '580 application) and U.S. patent application Ser. No. 18/103,169 (the '169 application), each of which is incorporated into this disclosure in its entirety and for all purposes. The device 102 may include a handle assembly 106, a pumping assembly 110, a tissue engagement portion 108, an outlet tube 132, and an electrical cable 134. The device 102 further includes an inlet port 138 which enables the introduction of other devices and materials to the distal end of the device 102 as will be described in greater detail below.

In FIG. 1B, the tip of the device 102 is shown in an exploded view. The pumping assembly 110 is located within a catheter body 104 and includes a stator 114, a locating element 118, a rotor 116, and a distal sleeve 120. A torque member 122 is rotationally connected to the rotor 116.

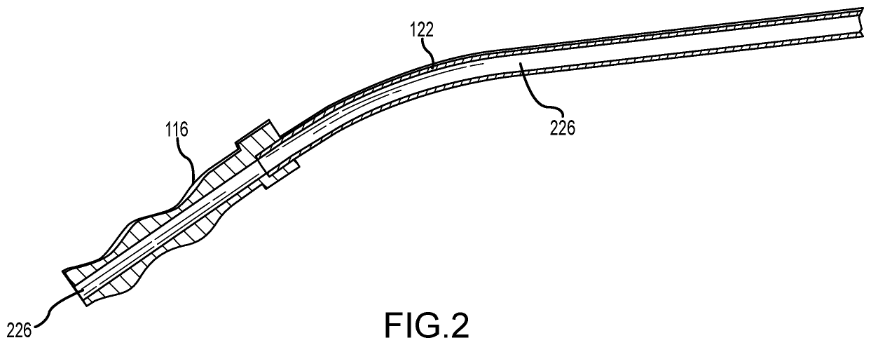

In FIG. 2, the rotor 116 and torque member 122 are shown in a section view and a center lumen 226 is shown. The center lumen 226 runs through the rotor 116 and through the torque member 122. The torque member 122 may be a torque coil or a tube that includes a hollow core which forms the center lumen 226.

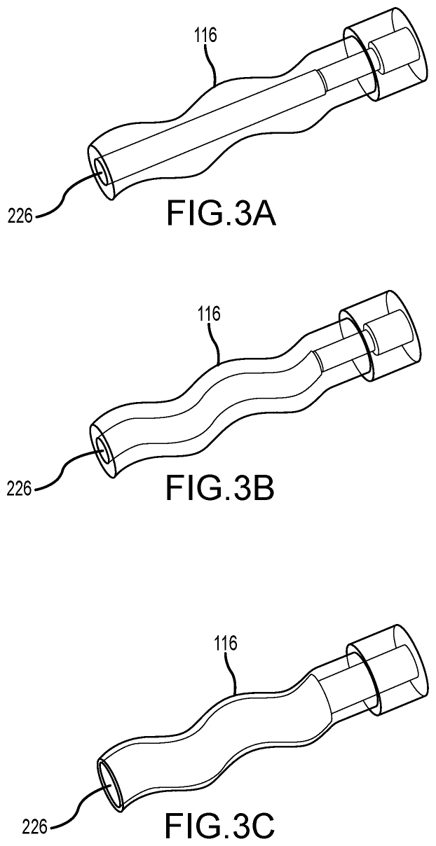

In FIGS. 3A-3C, several implementations of the rotor 116 with a central lumen 226 are shown. In FIG. 3A, the central lumen 226 runs straight through the rotor 116. The eccentricity and cross-sectional diameter of the rotor 116 are optimized so that central lumen 226 can run straight without twisting. This may enable the passage of devices such as guidewires and imaging elements which are difficult to twist in a helical pattern.

In FIG. 3B, the central lumen 226 is a helical pattern that follows the helical path of the rotor 116. In FIG. 3C, the central lumen 226 is also a helical pattern but is enlarged such that an appropriately sized guidewire or other device may not need to follow a helical path and can instead run straight through the central lumen 226. For example, if the rotor 116 cross sectional diameter is. 0.100″, the central lumen 226 diameter can be 0.090″ such that the wall thickness of the rotor 116 in this portion is about 0.005″. Even though the rotor 116 follows a helical path, a guidewire with a 0.035″ diameter can pass through the central lumen 226 straight without forming a helix. In some implementations, the rotor 116 can be formed by a hypotube that has been bent into the helical shape. In other implementations, the rotor 116 can be molded from thermoplastic or machined from thermoplastics or metals.

In operation, the device 102 can be used as described in greater detail in the '580 application and the '169 application. The device 102 can further be used as described below. The central lumen 226 can extend through the rotor 116, torque member 122, and other components until it reaches the inlet port 138. The user can introduce devices and fluids to the inlet port 138 such that they are delivered to the distal end of the rotor 116. Several examples are discussed below but are not intended to limit which devices and materials may be introduced or removed through the central lumen.

In some implementations, a guidewire may be inserted into the inlet port 138 and through the device 102. Alternatively, the guidewire may be backloaded into the central lumen 226 in the rotor 116 and out the inlet port 138. Such a configuration may enable the device 102 to be tracked over a guidewire to a desired anatomical location. Imaging elements such as OCT wires and IVUS catheters can be inserted through the central lumen 226 to provide visualization of the vessel prior to, during, or after device 102 operation. In some implementations, a wire may be extended through the central lumen 226 and as the rotor 116 spins, the tip of the wire may be configured to disrupt the clot by spinning and macerating the clot.

In some implementations, contrast may be injected through the inlet port 138. Similarly, thrombolytics can be delivered through the inlet port 138 and central lumen 226 in order to dissolve clots.

In some implementations, saline may be infused in the inlet port 138 at a given pressure or flow rate. This can be useful to reduce the amount of removed blood from the body by the device by infusing saline so the saline is pumped out by the pumping assembly 110 rather than blood. In other embodiments, the saline may be infused at high pressures and flow rates and used to disrupt clot at the tip of the rotor 116. This may be useful in combination with the tissue engagement portion 108 to ingest thrombus.

In some implementations, the central lumen 226 can be used to aspirate fluid. For example, blood could be aspirated to pull clot to the front of the device 102 and draw it into the tissue engagement portion 108. In some implantations, the device 102 can monitor the pressure and flow rate of the aspirated fluid to determine if the tip of the central lumen 226 is clogged with clot. This can be useful for determining when the device 102 is engaged with clot and therefore only turn on the pumping assembly 110 when clot is engaged at the distal tip of the device.

In some implementations, the inlet port 138 may further include or be connected to a hemostasis valve such as a Tuohy Borst adaptor that allows the introduction of fluid and devices simultaneous.

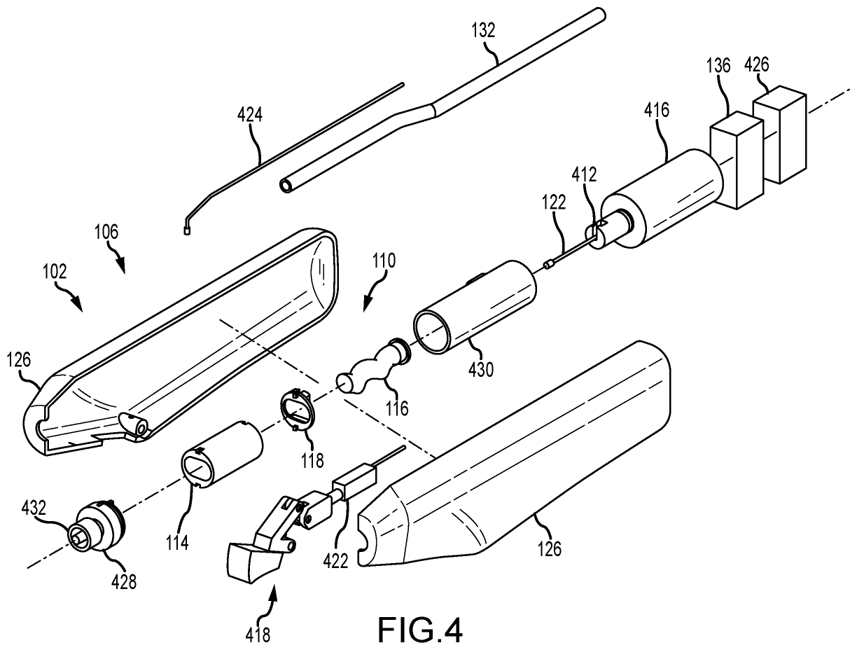

In FIG. 4, the device 102 has the pumping assembly 110 within the handle assembly 106 and, in certain implementations, the handle assembly 106 may function as an aspiration pump for aspiration catheters. The handle assembly 106 shown in FIG. 4 includes handle shells 126 collectively forming a handle body and each of a pumping assembly 110, a throttle assembly 418, a motor 416, a torque member 122, a motor adaptor 412, a controller 136, a battery 426, and a pressure sensor 424 disposed within the handle body. The handle assembly 106 further includes a catheter connector 432 in fluid communication with a first volume within the handle body (e.g., a volume distal the pumping assembly 110 or otherwise between the pumping assembly 110 and the patient) and an outlet tube 132 in fluid communication with a second volume within the handle body (e.g., a volume proximal the pumping assembly 110 or otherwise “upstream” of the patient). The pumping assembly 110 shown is a progressive cavity pump which includes a proximal housing 430 and a distal cap 428 which contain a stator 114, a rotor 116, and a locating element 118. The pumping assembly 110 works by rotation of the rotor 116 within the stator 114 to create a series of cavities that are formed at the distal end of the stator 114 and translate proximally through the pumping assembly 110 as the rotor 116 is rotated. This operation can draw fluid and other materials through the pumping assembly 110 and discharged into the outlet tubing 132. The operation of the pumping assembly 110 can also be reversed by turning the rotor 116 the opposite direction to discharge fluid into the distal cap 428 and possibly into a catheter. The device 102 shown includes a controller 136 and battery 426 within the handle assembly 106 such that no external capital equipment is required. Alternatively, the device 102 can be controlled and powered by one or more external cables that are supply control and power to the device 102.

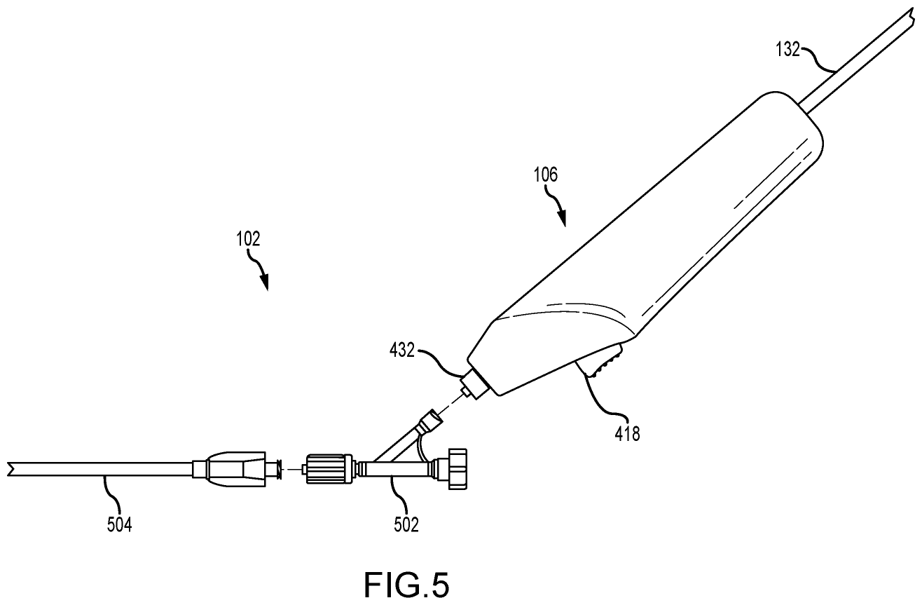

In FIG. 5, the device 102 is shown with an aspiration catheter 504 and a hemostasis valve 502. The handle assembly 106 which includes the pumping assembly 110 can be connected to the hemostasis valve 502 as shown using the catheter connector 432 which may be a luer connector or any other suitable connection. The aspiration catheter 504 can then be connected to the hemostasis valve 502. In this manner, the handle assembly 106 may function as an aspiration pump for the aspiration catheter 504 with the hemostasis valve 502 enabling the incorporation of a guidewire or other devices. In other implementations, the handle assembly 106 can be directly connected to the aspiration catheter 504 or can be connected by any number of other fittings or valves or connectors.

In operation, the aspiration catheter 504 can be placed into a blood vessel or other body cavity requiring aspiration. For example, the aspiration catheter 504 may be placed in the iliac vein of a patient for the removal of a deep vein thrombosis clot. Any number of other clinical applications are contemplated including but not limited to pulmonary embolism ischemic stroke, and cardiac thrombosis. The aspiration pump within the handle assembly 106 can be activated by activating the throttle assembly 418. The aspiration created can withdraw fluid and other material such as clot from the lumen of the aspiration catheter 504 and into the pumping assembly 110 and discharged into the outlet tubing 132. The outlet tubing 132 can be connected to a waste container or any number of filters or assemblies. In some implementations, the removed blood can be filtered and reintroduced into the patient to reduce blood loss of the patient. In some implementations, the pumping assembly 110 or the aspiration catheter 504 is primed with fluid such as saline prior to creating significant vacuum forces.

Typical aspiration catheter systems include a pump which is large and located several feet away from the patient. These systems generate a large amount of vacuum in a container that serves as a vacuum reservoir which can be applied to the aspiration catheter through valves. An advantage of including the pumping assembly 110 within the handle assembly 106 and close to the end of the aspiration catheter 504 is faster response times and higher vacuum levels at the tip of the aspiration catheter 504 due to less head loss from tubing and connectors. Without a large vacuum reservoir and significantly less tubing, the device 102 has a lower ‘dead volume’ than traditional aspiration systems. The aspiration is only applied when the pumping assembly 110 is activated. Additionally, progressive cavity pumps beneficially have discrete pumping volumes based on the number of rotations of the rotor 116. This provides further control of the fluid removed by the device 102.

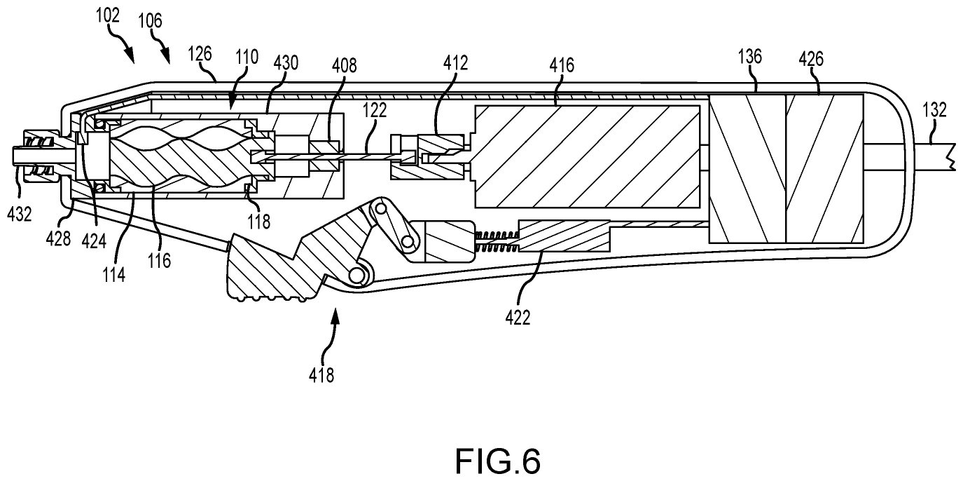

In FIG. 6, a section view of the handle assembly 106 is shown. The pumping assembly 110 is connected to the motor 416 through a motor adaptor 412 and a torque member 122. The torque member 122 can be flexible cable such as a torque cable, a drive cable, a torque coil, a wire rope, or any other suitable construction. A flexible cable may beneficially allow some movement of the distal connection that is not purely rotational as the rotor 116 translates side-to-side as it rotates. In some implementations, the torque member 122 can be connected to a point further distal on the rotor 116 such that the side-to-side movements create a smaller angle of movement and thus require less flexibility. The torque member 122 can be comprised of any number of other constructions as well such as laser cut tubes or rods and universal couplers. A sealing member 408 is included to allow the torque member 122 to rotate and maintain sealing of the fluid within the proximal housing 430. The distal cap 428 is connected to the distal end of the proximal housing 430 and includes the catheter connector 432, forming an internal volume of the handle assembly 106 distal the pumping assembly 110. A locating element 118 is included that provides the rotor 116 a bearing surface and hardstop as it rotates which prevents the rotor 116 from moving distally as it pumps fluid proximally into the pumping assembly 110. A similar feature is shown in the proximal housing 430 that prevents the rotor 116 from moving proximally as it pumps fluid distally out of the pumping assembly 110. The locating element 118, the proximal housing 430, the distal cap 428, and the stator 114 can additionally have features which provide rotational alignment of the components relative to one another. As the rotor 116 imparts rotational forces on the stator 114, the alignment features can prevent the stator 114 from rotating relative to the proximal housing 430. The outlet tubing 132 (or similar outlet conduit) is connected to the proximal housing 430 in communication with an internal volume of the handle assembly 105 proximal the pumping assembly 110 and provides an outlet for the discharged fluid and material. A pressure sensor 424 is shown in the distal cap 428 and is electronically connected to the controller 136. In this manner, the controller 136 can adjust the activation of the pumping assembly 110 based on the pressure that is sensed within the distal cap 428. The throttle assembly 418 includes a throttle sensor 422 which may be a displacement potentiometer or any other sensor for providing feedback to the controller 136 about the throttle assembly 418 movement. For example, the throttle sensor 422 can be held in place by features in the handle shell 126 and as the throttle assembly 418 is depressed the potentiometer can adjust the voltage in the throttle sensor 422 which can be interpreted by the controller 136.

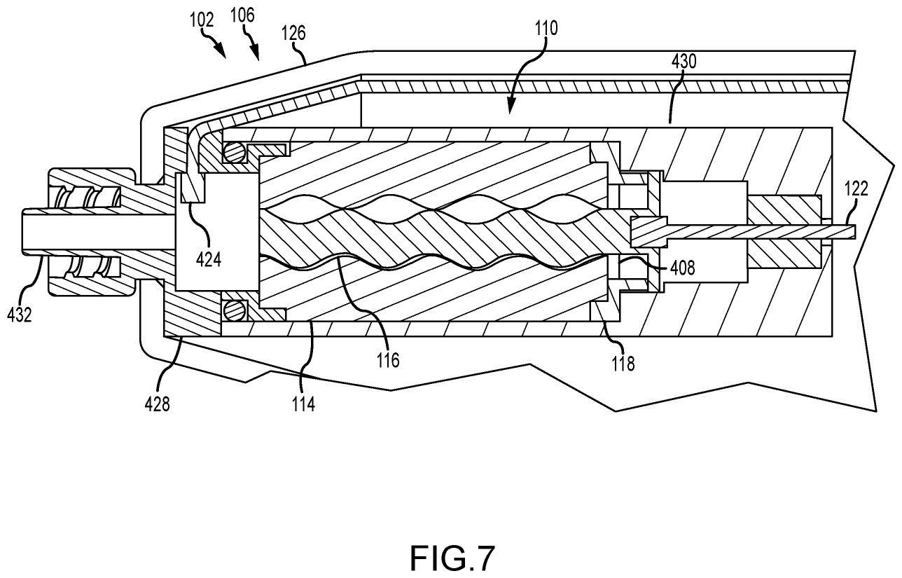

In FIG. 7, another implementation of the pumping assembly 110 is shown. The rotor 116 is smaller in diameter and has a shorter helical pitch that matches the geometry in the stator 114. This implementation is a 2-stage progressive cavity pump since there are at least 2 fully scaled cavities within the pumping assembly 110 at any given time. Such an implementation my provide higher suction forces by increasing the sealing amount of each cavity. Additionally, each cavity is smaller than the cavities shown in FIG. 6 and therefore may provide higher resolution with regards to the amount of fluid removed.

Additionally, as will be discussed in greater detail below, the motor 416 and controller 136 can be configured to apply different patterns of movement to create different vacuum profiles. For example, the motor 416 can be pulsed on and off to create a pulsatile vacuum at the tip of the catheter body 104. Pulsatile vacuum has been shown to improve clot ingestion. In some implementations, the motor 416 can be spun to draw in fluid and generate vacuum. Then the motor 416 direction can be reversed to reduce the vacuum level to zero or to a lower level. These patterns and cycles can happen many times a second to create rapid pulsatile vacuum profiles. Pressure sensors 424 can also be used to create different desired vacuum or flow rate patterns.

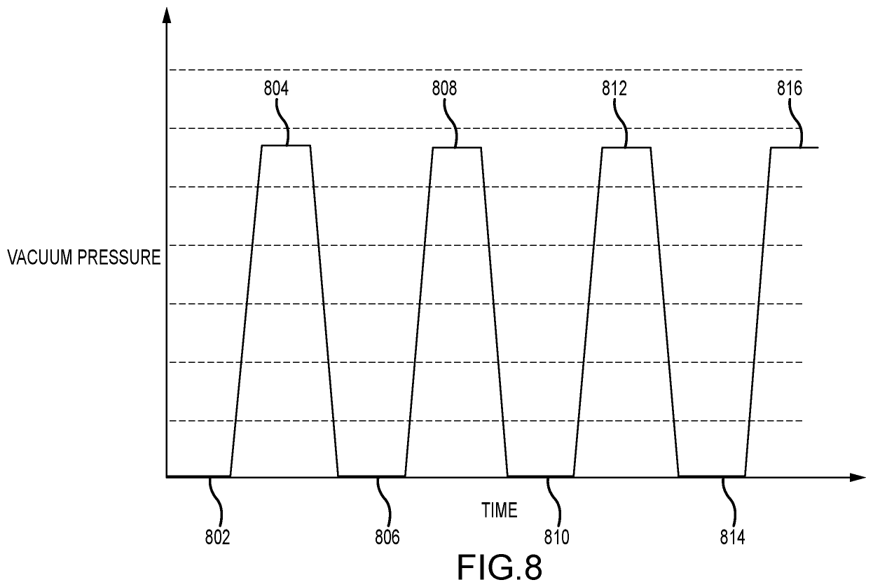

In FIG. 8, an implementation of a vacuum pressure profile is shown in a graph. The vacuum pressure shown in the y-axis can be the pressure within the distal cap 428 or within any part of the aspiration catheter 504. A first vacuum pressure 802 is at zero before the pumping assembly 110 has been activated. Upon activation of the pumping assembly 110, a second vacuum pressure 804 is achieved within the aspiration catheter 504. In certain implementations, the second vacuum pressure 804 can be from and including about 1 inHg to and including about 30 inHg or about 27 inHg. The pumping assembly 110 can then be turned off or reversed to reduce to a third vacuum pressure 806. Reversal of the pumping assembly 110 can be controlled by the controller 136 and can be done to a predefined number of rotations or can be done while monitoring the pressure sensor 424 to a specific pressure level. Reversing the pumping assembly 110 can allow a small amount of fluid to return to the aspiration catheter 504 where vacuum pressure is accumulated. This can reduce the vacuum pressure or even create a positive pressure in the aspiration catheter 504. In some implementations, at least a small amount of vacuum pressure can be maintained to ensure fluid and material do not come out of the aspiration catheter 504. This process can be repeated cyclically through a fourth vacuum pressure 808, a fifth vacuum pressure 810, a sixth vacuum pressure 812, a seventh vacuum pressure 814, and an eighth vacuum pressure 816. Although the lower vacuum pressures and upper vacuum pressures are shown returning to similar levels on the graph, it is not necessarily required and any number of profiles may be considered. Additionally, the amount of time at each vacuum pressure and the amount of ramp time between each vacuum need not necessarily be the same for each cycle. In some implementations, the controller 136 can adjust the profile to achieve predetermined vacuum pressures as measured by the pressure sensor 424 and as shown in the graph in FIG. 8. In other implementations, the controller 136 can adjust the pumping assembly 110 speed only or the flow rate only and not achieve predetermined vacuum pressures. Any number of other control methods by the controller 136 are contemplated. In certain implementations, the frequency of the cyclic vacuum pressure in FIG. 8 can be from and including about 0.01 Hz to and including about 2000 Hz, or about 10 Hz.

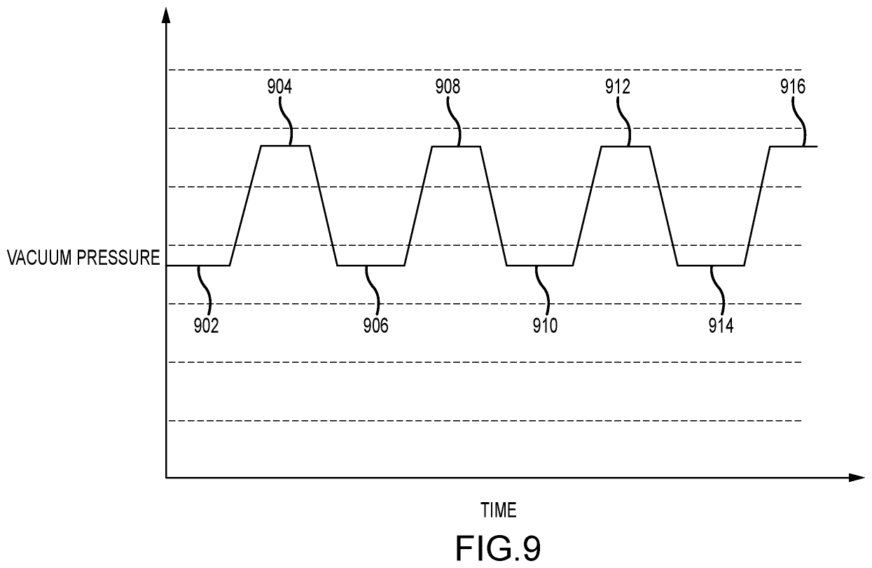

In FIG. 9, another implementation of the vacuum pressure profile is shown in a graph. In this implementation, the lower vacuum pressures 902, 906, 910, and 914 are significantly above zero and alternate with the higher vacuum pressures 904, 908, 912, and 916. This may keep vacuum on the aspiration catheter 504 at all times while still cycling the vacuum pressure. Beneficially, this may prevent any distal movement of fluid out of the aspiration catheter 504 and reduce the risk of clot embolizing in the blood stream of the patient or prevent blood from exiting the aspiration catheter 504.

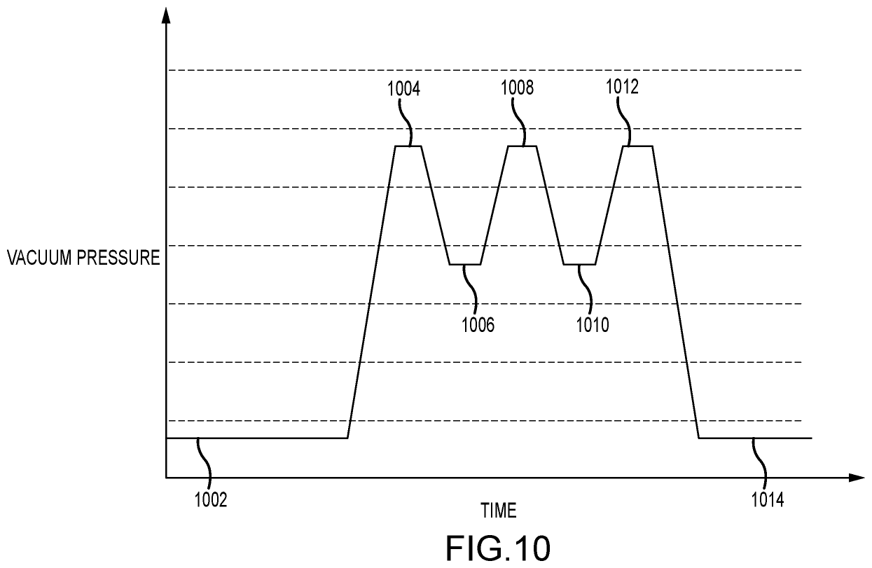

In FIG. 10, another implementation of the vacuum pressure profile is shown in a graph. In this implementation, the pumping assembly 110 can be turned at slow speeds to remove lower amounts of blood until a clot is encountered at the tip. The first vacuum pressure 1002 shows a low level of vacuum in the aspiration catheter 504 as blood is slowly removed. Once clot is engaged at the tip of the aspiration catheter 504, the aspiration pressure can increase to a second vacuum pressure 1004. The controller 136 can sense this either through a pressure sensor 424, an increase in torque of the rotor 116, or other methods and then a different motor profile can be used to increase the aspiration force on the clot. For example, the controller 136 can implement a cyclic vacuum profile as shown (e.g., by alternating between high vacuum pressures 1004, 1008, and 1012 and intermediate vacuum pressures 1006 and 1010). When the clot is fully ingested into the aspiration catheter 504, the controller 136 can sense the reduced vacuum pressure (e.g., low vacuum pressure 1014) and change the vacuum profile back to a relatively low flow vacuum.

The foregoing examples of vacuum pressure profiles are generally described in the context of handheld devices, such as those shown in FIGS. 4-7, in which a progressive cavity pump is included in a handle assembly from which a catheter extends. However, this disclosure contemplates that pulsatile vacuum profiles and related concepts may be readily incorporated into other devices, such as the device of FIG. 1, in which the progressive cavity pump is integrated into a distal portion of a catheter. In such devices, the distal portion of the catheter may support or include a pressure sensor in communication with a controller to measure pressure in a volume distal the progressive cavity pump. In certain implementations, the volume may be within a portion of the catheter or a tip that extends distally beyond the progressive cavity pump. In other implementations, the volume may correspond to a volume external the catheter (e.g., within the blood lumen of the patient) but near the distal extent of the progressive cavity pump. Regardless of the specific volume being monitored by the pressure sensor, the pressure measurements obtained by the pressure sensor may be provided as feedback to the controller of the device. The controller may then activate the motor to drive the progressive cavity pump rotor according to any of the vacuum pressure profiles discussed above or variations of such profiles within the scope of this disclosure.

In some implementations, the handle assembly 106 can be further split into a disposable subassembly and a reusable subassembly. For example, the disposable subassembly can include the pumping assembly 110 and outlet tubing 132 which have direct contact with bodily fluids. A reusable assembly can include the motor 416, controller 136, and battery 426 or power supply. These subassemblies can be connected such that the motor 416 can supply torque and rotational energy to the pumping assembly 110. Electrical and mechanical connections between the two subassemblies can be further integrated.

In some implementations, the pumping assembly 110 is not within a handle assembly 106 but in a dedicated aspiration pump that is connected to the aspiration catheter 504 with a series of tubes. The user can control the pumping assembly 110 by smaller handheld assemblies which communicate electronically or physically with the pumping assembly 110. Such an implementation of using a progressive cavity pump as a pumping assembly 110 still provides many of the benefits over traditional aspiration pumps.

Much description has been given to thrombectomy procedures where clot and thrombus is removed but that is not intended to limit the scope or use of the device 102 to such procedures. For the removal of doubt, the terms clot and thrombus can be considered interchangeable with any material that is being removed. The device 102 can have a variety of shapes and sizes serving as a platform for any type of thrombectomy, embolectomy, or foreign body, calculi or tissue removal in any part of the body or vessel. This could include but not limited to cerebral thrombi causing ischemic strokes, deep venous thrombosis both acute and chronic, pulmonary emboli, dural sinus thrombosis, controlled aspiration of tissue and/or fluid during surgery of the ventricular system or cerebrum, removal of liquid embolic agent, clotted hemodialysis grafts, peripheral arterial thromboemboli, including the mesenteric and peripheral vascular tree, peripheral arterial occlusion, critical limb ischemia (CLI), chronic total occlusion (CTO) and stone removal. The device 102 may also be used for debulking procedures for the removal of tumor and other cancerous materials.

Any number of other suitable applications may use such a device 102 for the removal of a tissue, foreign body, calculi or other objects within a tubular contained space or even within non-tubular or non-contained spaces. In some implementations, the device 102 may be used for removal of tissue during small port laparoscopic procedures include biopsies or removal of malignant tissue.

The names and labels applied to the various components and parts should not be considered limiting to the scope of the invented device and method.

Although implementations of various methods and devices are described herein in detail with reference to certain versions, it should be appreciated that other versions, implementations, methods of use, and combinations thereof are also possible. Therefore, the spirit and scope of the appended claims should not be limited to the description of the implementations contained herein.

1.-20. (canceled)

21. A device, comprising:
a handle coupleable to a catheter;
a motor;
a progressive cavity pump including a rotor operably coupled to the motor such that actuation of the motor causes rotation of the rotor, wherein, when the handle is coupled to the catheter, the progressive cavity pump is in fluid communication with the catheter; and
a controller communicatively coupled to the motor and configured to activate the motor to produce a pulsatile vacuum using the progressive cavity pump.

22. The device of claim 21, further comprising a pressure sensor configured to measure pressure of the pulsatile vacuum produced by the progressive cavity pump, wherein the controller is further configured to activate the motor to produce the pulsatile vacuum based on pressure measurements obtained using the pressure sensor.

23. The device of claim 22, further comprising the catheter, wherein the progressive cavity pump is disposed in a distal tip of the catheter and the pressure sensor is configured to measure pressure distal the progressive cavity pump.

24. The device of claim 22, wherein the handle includes a handle body and the progressive cavity pump is disposed within the handle body and configured to transfer fluid between a first volume within the handle body and a second volume within the handle body, wherein the first volume is disposed between the progressive cavity pump and one of the catheter and a catheter connection and wherein the pressure sensor is disposed within the first volume.

25. The device of claim 21, wherein the controller is configured to activate the motor for a first activation period during which the rotor is rotated in a first direction and a second activation period during which the rotor is rotated in a second direction opposite the first direction.

26. The device of claim 21, wherein the controller is configured to activate the motor for a first activation period during which the rotor is rotated and a second activation period during which the rotor is stopped.

27. The device of claim 21, wherein the controller is configured to activate the motor based on a vacuum pressure profile, and wherein the vacuum pressure profile includes a first period of a first vacuum pressure produced by the progressive cavity pump and a second period of a second vacuum pressure produced by the progressive cavity pump, the second vacuum pressure being different than the first vacuum pressure.

28. The device of claim 27, wherein the first vacuum pressure is positive and the second vacuum pressure is zero.

29. The device of claim 27, wherein each of the first vacuum pressure and the second vacuum pressure is positive.

30. The device of claim 27, wherein the vacuum pressure profile further includes a third vacuum pressure between the first vacuum pressure and the second vacuum pressure.

31. The device of claim 21 further comprising a hollow torque member including a first lumen, wherein the rotor defines a second lumen extending through the rotor and in communication with the first lumen.

32. A method of removing occlusive material within a blood lumen of a patient, the method comprising:
disposing a catheter coupled to a handle assembly within the blood lumen; and
generating a pulsatile vacuum using a progressive cavity pump in fluid communication with the catheter and in a volume distal the progressive cavity pump.

33. The method of claim 32, wherein generating the pulsatile vacuum includes actuating a motor operably coupled to a rotor of the progressive cavity pump for a first activation period during which the rotor is rotated in a first direction and a second activation period during which the rotor is rotated in a second direction opposite the first direction.

34. The method of claim 32, wherein generating the pulsatile vacuum includes actuating a motor operably coupled to a rotor of the progressive cavity pump for a first activation period during which the rotor is rotated and a second activation period during which the rotor is stopped.

35. The method of claim 32, wherein:
generating the pulsatile vacuum includes actuating a motor operably coupled to a rotor of the progressive cavity pump,
the motor is communicatively coupled to a controller configured to activate the motor based on a vacuum pressure profile,
generating the pulsatile vacuum further includes the controller activating the motor according to the vacuum pressure profile and pressure measurements obtained from a pressure sensor, and
the vacuum pressure profile includes a first period of a first vacuum pressure in the volume and a second period of a second vacuum pressure in the volume, the second vacuum pressure being different than the first vacuum pressure.

36. The method of claim 35, wherein the first vacuum pressure is positive and the second vacuum pressure is zero.

37. The method of claim 35, wherein each of the first vacuum pressure and the second vacuum pressure is positive.

38. The method of claim 35, wherein the vacuum pressure profile further includes a third vacuum pressure between the first vacuum pressure and the second vacuum pressure.

39. The method of claim 32, wherein the progressive cavity pump is disposed in a distal portion of the catheter.

40. The method of claim 32, wherein the progressive cavity pump is disposed within a handle body of a handle assembly coupled to the catheter.
